(19) United States
(12) Patent Application Publication
(10) Pub. No.: US 2013/0274284 A1
(43) Pub. Date: Oct. 17, 2013

(54) PREPARATION OF PRASUGREL HYDROCHLORIDE

(71) Applicants: Raghupathi Reddy Anumula, Hyderabad (IN); Goverdhan Gilla, Chandanagar (IN); Sampath Aalla, Quthbullapur (IN); Dattatray Shamrao Metil, Navi Mumbai (IN); Srinivas Kurella, Nalgonda (IN); Kavitha Charagondla, Nalgonda (IN)

(72) Inventors: Raghupathi Reddy Anumula, Hyderabad (IN); Goverdhan Gilla, Chandanagar (IN); Sampath Aalla, Quthbullapur (IN); Dattatray Shamrao Metil, Navi Mumbai (IN); Srinivas Kurella, Nalgonda (IN); Kavitha Charagondla, Nalgonda (IN)

(73) Assignees: DR. REDDY'S LABORATORIES, INC., Bridgewater, NJ (US); Dr. Reddy's Laboratories Ltd., Hyderabad (IN)

(21) Appl. No.: 13/814,596

(22) PCT Filed: Aug. 2, 2011
(86) PCT No.: PCT/US11/46234

§ 371 Date: Jul. 8, 2013

Related U.S. Application Data
(60) Provisional application No. 61/390,234, filed on Oct. 6, 2010.
(60) Provisional application No. 61/476,450, filed on Apr. 18, 2011.

(30) Foreign Application Priority Data
Aug. 6, 2010 (IN) 2258/CHE/2010
Mar. 1, 2011 (IN) 611/CHE/2011

(51) Int. Cl. C07D 495/04

(52) U.S. Cl. CPC C07D 495/04
USPC 514/301; 546/114

(57) ABSTRACT

The present application relates to process for the preparation of prasugrel, its pharmaceutically acceptable salts, and its intermediates.

Aspects of the present application relate to processes for the preparation of prasugrel hydrochloride and pharmaceutical compositions comprising prasugrel hydrochloride.

The drug compound having the adopted name “prasugrel hydrochloride” can be represented by structural formula (I), and it is a thienopyridine class inhibitor of platelet activation and aggregation mediated by the P2Y₁₂ adenosine diphosphate (ADP) receptor.

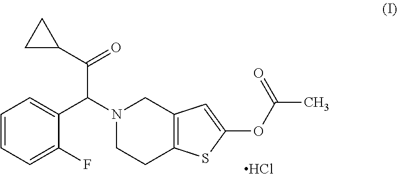 [SMILES: CC(=O)Oc1cc2c(s1)CCN(C(C(=O)C1CC1)c1ccccc1F)C2 ; Cl]

Prasugrel hydrochloride is the hydrochloride salt of a racemate of prasugrel. A chemical name for prasugrel hydrochloride is 5-[(1RS)-2-cyclopropyl-1-(2-fluorophenyl)-2-oxoethyl]-4,5,6,7-tetrahydrothieno[3,2-c]pyridin-2-yl acetate hydrochloride, and it is the active ingredient in Effient® tablets approved for the treatment of acute coronary syndrome.

U.S. Pat. No. 5,288,726 discloses prasugrel, or a tautomer thereof, or a pharmaceutically acceptable salt thereof, pharmaceutical composition thereof and a method for the treatment or prophylaxis of thrombosis or embolisms comprising administering a mammal an effective amount of the compound(s) thereof.

The processes for preparing Prasugrel or Prasugrel hydrochloride have been described in a number of references, including: (I) U.S. Pat. No. 5,288,726; (II) U.S. Pat. No. 6,693,115; (III) U.S. Pat. No. 5,874,581; (IV) U.S. Patent Application Publication No. 2009/0203729 A1; (V) International Application Publication No. WO 2008/108291A1; (VI) International Application Publication No. WO 2009/062044 A1; (VII) International Application Publication No. WO 2009/066326 A1; (VIII) International Application Publication No. WO 2009/122440A1 and (IX) International Application Publication No. WO 2010/070677 A2.

Like any synthetic compound, prasugrel can contain extraneous compounds or impurities that can come from many sources. They can be unreacted starting materials, by-products of the reaction, products of side reactions, or degradation products. Impurities in prasugrel or any active pharmaceutical ingredient (API) are undesirable, and, in extreme cases, might even be harmful to a patient being treated with a dosage form of the API in which a sufficient amount of impurities is present.

There remains a need for a short, industrially scalable, cost-effective, and environmentally-friendly process for the preparation of prasugrel hydrochloride resulting in a stable polymorphic form of prasugrel hydrochloride with high purity which can be easily formulated.

### SUMMARY

In an aspect, the application provides a process for preparing prasugrel, comprising:
a) reacting 5,6,7,7a-tetrahydro-thieno[3,2-c]pyridin-2(4H)-one of formula (II), or a salt thereof with 2-bromo-1-cyclopropyl-2-(2-fluorophenyl)ethanone of formula (III), in the presence of an inorganic base and in a solvent to produce 5-(2-cyclopropyl-1-(2-fluorophenyl)-2-oxoethyl)-5,6,7,7a-tetrahydrothieno[3,2-c]pyridin-2(4H)-one of formula (IV);
b) acetylating in situ the obtained 5-(2-cyclopropyl-1-(2-fluorophenyl)-2-oxoethyl)-5,6,7,7a-tetrahydrothieno[3,2-c]pyridin-2(4H)-one of formula (IV) in step a), into prasugrel by treating with an acetylating agent in the presence of an organic base; and
c) optionally converting the prasugrel of step b), into prasugrel hydrochloride in the presence of a source of hydrogen chloride in an organic solvent.

### BRIEF DESCRIPTION OF THE DRAWING

FIG. 1 depicts PXRD pattern of prasugrel hydrochloride obtained by the procedure of Example 4.

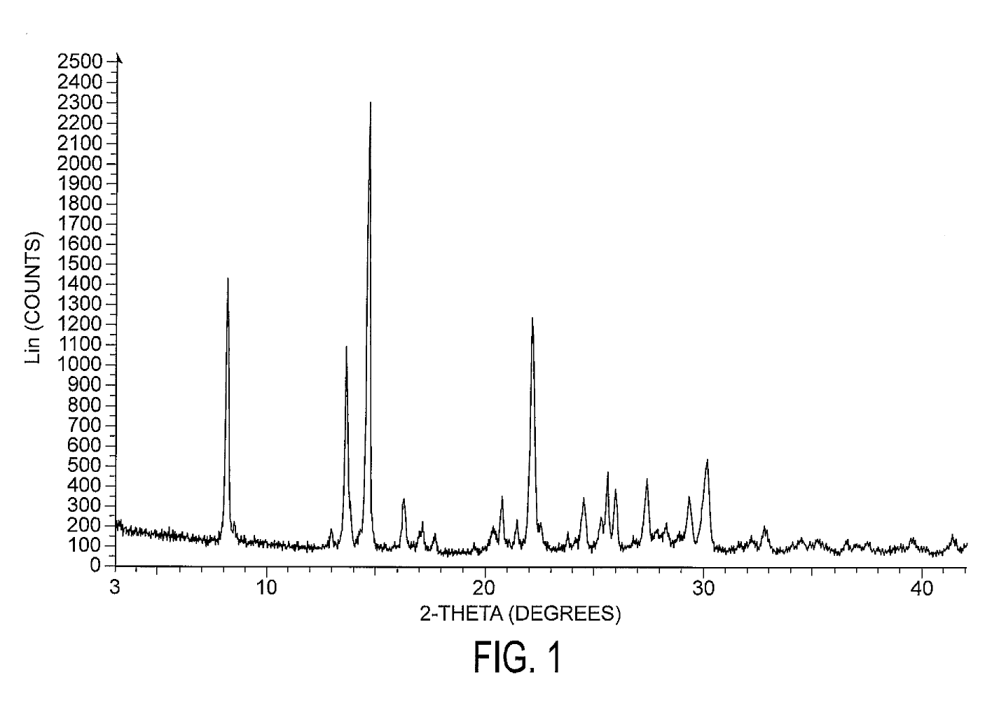

### DETAILED DESCRIPTION

In an aspect, the application provides a process for preparing prasugrel, comprising:
a) reacting 5,6,7,7a-tetrahydro-thieno[3,2-c]pyridin-2(4H)-one of formula (II), or a salt thereof with 2-bromo-1-cyclopropyl-2-(2-fluorophenyl)ethanone of formula (III), in the presence of an inorganic base and in a solvent to produce 5-(2-cyclopropyl-1-(2-fluorophenyl)-2-oxoethyl)-5,6,7,7a-tetrahydrothieno[3,2-c]pyridin-2(4H)-one of formula (IV);
b) acetylating in situ the obtained 5-(2-cyclopropyl-1-(2-fluorophenyl)-2-oxoethyl)-5,6,7,7a-tetrahydrothieno[3,2-c]pyridin-2(4H)-one of formula (IV) in step a), into prasugrel by treating with an acetylating agent in the presence of an organic base; and
c) optionally converting the prasugrel of step b), into prasugrel hydrochloride in the presence of a source of hydrogen chloride in an organic solvent.

In embodiments of step a), the reaction of the starting material used i.e., 5,6,7,7a-tetrahydro-thieno[3,2-c]pyridin-2(4H)-one of formula (II), or a salt thereof with 2-bromo-1-cyclopropyl-2-(2-fluorophenyl)ethanone of formula (III), can be carried out in presence of a base and in a solvent to produce 5-(2-cyclopropyl-1-(2-fluorophenyl)-2-oxoethyl)-5,6,7,7a-tetrahydrothieno[3,2-c]pyridin-2(4H)-one of formula (IV) or a salt thereof.

In embodiments of step a), the starting material used i.e., 5,6,7,7a-tetrahydro-thieno[3,2-c]pyridin-2(4H)-one of formula (II), or a salt thereof, which is one of the starting materials for the preparation of prasugrel, may be prepared according to any of the processes disclosed in the art. Optionally, the compound of formula (II) may be converted into its acid-addition salt by reacting it with a pharmaceutically acceptable acid. Examples of such acids include but are not limited to: inorganic acids such as hydrochloric acid, sulfuric acid, phosphoric acid, hydrobromic acid, or the like; and organic acids such as oxalic acid, maleic acid, fumaric acid, malic acid, tartaric acid, citric acid, benzoic acid, or the like. In embodiments of step a), the starting material used i.e., 2-bromo-1-cyclopropyl-2-(2-fluorophenyl)ethanone of formula (III), which is one of the starting materials for the preparation of prasugrel, may be prepared according to any of the processes disclosed in the art.

In embodiments of step a), compound of formula (II) or a salt thereof and compound of formula (III) can be reacted in the mole ratio of 1:0.8 to 1:1.20 respectively. In one embodiment, they are reacted in the mole ratio of 1:1 respectively.

In embodiments of step a), the compound of formula (II), or a salt thereof, and the compound of formula (III), can be reacted in the presence of an inorganic base to produce 5-(2-cyclopropyl-1-(2-fluorophenyl)-2-oxoethyl)-5,6,7,7a-tetrahydrothieno[3,2-c]pyridin-2(4H)-one, compound of formula (IV). Inorganic bases that are useful in the reaction include, but are not limited to; inorganic bases, such as, for example, alkali metal hydrides, such as, for example, lithium hydride, sodium hydride, potassium hydride, or the like; sodamide; n-butyl lithium; lithium diisopropylamide; alkali metal hydroxides, such as, for example, lithium hydroxide, sodium hydroxide, potassium hydroxide, or cesium hydroxide; alkaline metal hydroxides, such as, for example, barium hydroxide, magnesium hydroxide, calcium hydroxide, or the like; alkali metal carbonates, such as, for example, sodium carbonate, potassium carbonate, lithium carbonate, cesium carbonate, or the like; alkaline earth metal carbonates, such as, for example, magnesium carbonate, calcium carbonate, or the like; alkali metal bicarbonates, such as, for example, lithium bicarbonate, sodium bicarbonate, potassium bicarbonate, or the like; and ion exchange resins including resins bound to ions, such as, for example, sodium, potassium, lithium, calcium, magnesium, substituted or unsubstituted ammonium ions, or the like; or any other suitable base. However the compound of formula (II), or a salt thereof, and the compound of formula (III), can also be reacted in the presence of an organic bases such as for example, amines, e.g., triethylamine, N,N-diethylethanolamine, 4-ethylmorpholine, 1,4-diazabicyclo[2.2.2]-octane, N-methyl morpholine, diisopropylamine diisopropylethylamine, pyridine, or the like.

In embodiments of step a), the compound of formula (II), or a salt thereof, and the compound of formula (III), can be reacted in the presence of a solvent, to produce 5-(2-cyclopropyl-1-(2-fluorophenyl)-2-oxoethyl)-5,6,7,7a-tetrahydrothieno[3,2-c]pyridin-2(4H)-one, compound of formula (IV). Suitable solvent can be any solvent which has no adverse effect on the reaction or on the reagents involved and that it can dissolve the reagents, at least to some extent. Examples of such solvents include an ether solvent, such as diethyl ether, diisopropyl ether, tetrahydrofuran, dioxane, or dimethoxyethane; a ketone solvent, such as acetone or methyl ethyl ketone, methyl isobutyl ketone or diethyl ketone; an ester solvent, such as ethyl acetate, propyl acetate or butyl acetate; an alcohol solvent such as methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, iso-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, or C₁-C₆ alcohols; a nitrile solvent, such as acetonitrile, propionitrile; amides, such as N,N-dimethylformamide, N,N-dimethyl acetamide, N-methyl-2-pyrrolidone or hexamethyl phosphoric triamide; sulfoxides, such as dimethyl sulfoxide; water; or their mixtures thereof.

In embodiments of step a), the reaction of compound of formula (II), or a salt thereof, and the compound of formula (III), can be carried out at a temperature ranging from about 0° C. to about boiling point of the solvent. In one embodiment, the reaction can be carried out from about room temperature to about boiling point of the solvent. The time required for the reaction may also vary widely, depending on many factors, notably the reaction temperature and the nature of the reagents and solvent employed. However, provided that the reaction is effected under the conditions outlined above, a period of from about 1 hour to about 24 hours or longer is sufficient.

In embodiments of step a), the obtained 5-(2-cyclopropyl-1-(2-fluorophenyl)-2-oxoethyl)-5,6,7,7a-tetrahydrothieno[3,2-c]pyridin-2(4H)-one of formula (IV) in step a), can be used in the next step without isolation i. e., in situ. In embodiments of step a), the obtained 5-(2-cyclopropyl-1-(2-fluorophenyl)-2-oxoethyl)-5,6,7,7a-tetrahydrothieno[3,2-c]pyridin-2(4H)-one of formula (IV) in step a), can be isolated according to the procedures known in the art. In embodiments of step b), acetylating in situ the obtained 5-(2-cyclopropyl-1-(2-fluorophenyl)-2-oxoethyl)-5,6,7,7a-tetrahydrothieno[3,2-c]pyridin-2(4H)-one of formula (IV) in step a), into prasugrel by treating with an acetylating agent in an organic base.

In embodiments of step b), the compound of formula (IV) i.e. 5-(2-cyclopropyl-1-(2-fluorophenyl)-2-oxoethyl)-5,6,7,7a-tetrahydrothieno[3,2-c]pyridin-2(4H)-one, is reacted with an acetylating agent in presence of a base and a solvent to produce prasugrel. The acetylating agents, which can be used include, but are not limited to, acetic anhydride, acetyl chloride or any acetylating agent which produces prasugrel. In embodiments of step b), the compound of formula (IV), is reacted with an acetylating agent in presence of an organic base. Organic bases which can be used, but are not limited to: amines, e.g., triethylamine, N,N-diethylethanolamine, 4-ethylmorpholine, 1,4-diazabicyclo[2.2.2]-octane, N-methyl morpholine, diisopropylamine, diisopropylethylamine, pyridine, or the like. However inorganic bases can also be used in the acetylation step. Inorganic bases which can be used, but are not limited to: alkali metal or alkaline earth metal carbonates, hydrogen carbonates, hydroxides, oxides, carboxylates, alkoxides, and hydrides e.g., potassium carbonate, potassium hydrogen carbonate, potassium hydroxide, potassium acetate, potassium methoxide, sodium carbonate, sodium hydrogen carbonate, sodium hydroxide, sodium acetate, sodium methoxide, lithium carbonate, lithium hydrogen carbonate, lithium hydroxide, lithium acetate, lithium methoxide, barium hydroxide, calcium oxide, sodium hydride, potassium hydride or the like. In embodiments of step b), the compound of formula (IV), is reacted with an acetylating agent in presence of a base and a solvent to produce prasugrel. Suitable solvent can be any solvent which has no adverse effect on the reaction or on the reagents involved and that it can dissolve the reagents, at least to some extent. Examples of such solvents include; an ether solvent, such as diethyl ether, tetrahydrofuran or dioxane; a ketone solvent, such as acetone or methyl ethyl ketone; an ester solvent, such as ethyl acetate; a nitrile solvent, such as acetonitrile; amides, such as N,N-dimethylformamide, N,N-dimethylacetamide, N-methyl-2-pyrrolidone or hexamethylphosphoric triamide; and sulfoxides, such as dimethyl sulfoxide.

In embodiments of step b), the compound of formula (IV), is reacted with an acetylating agent in presence of a catalyst. In embodiments, catalysts that can be used may include, for example, 4-dialkylaminopyridines such as 4-dimethylaminopyridine, 4-diethylaminopyridine, 4-dipropylaminopyridine, etc. In embodiments of step b), acetylation of the compound of formula (IV) can be carried out at a temperature of from about −10° C. to about 60° C. In one embodiment, the temperature employed is from about −10° C. to about 30° C. The time required for the reaction may also vary widely, depending on many factors, notably the reaction temperature and the nature of the reagents and solvent employed. However, provided that the reaction is effected under the conditions outlined above, for a period of about 1 to about 24 hours or longer. In one embodiment, a time from about 1 hour to about 10 hours) will usually suffice. In embodiments of step b), prasugrel can be isolated from the reaction by cooling the reaction mass to a temperature from about 20° C. to −20° C. or by adding the anti solvents such as water.

In embodiments of step b), prasugrel produced in the reaction can be isolated using techniques such as decantation, filtration by gravity or suction, centrifugation, evaporation of solvent, or the like, and optionally washing the resulting solid with a solvent. In one embodiment, the washing is with the solvent used in the reaction. In embodiments of step b), the prasugrel obtained may be optionally dried according to the procedures known in the art.

In embodiments of step b), prasugrel that is isolated can be dried at suitable temperatures, such as from about 40° C. to about 100° C. and suitable time from about 1 hour to about 15 hours or longer, using drying equipment known in the art, such as a tray dryer, vacuum oven, air oven, fluidized bed dryer, spin flash dryer, flash dryer, or the like. Drying temperatures and times will be sufficient to achieve desired product purity.

In embodiments of step b), the prasugrel obtained can be optionally purified by any method known in the art such as recrystallization involving single solvent, mixture of solvents, or solvent-anti solvent technique; reprecipitation; slurring in a solvent; or chromatography to improve its chemical purity. Any of the solvents listed in step a), can be used for the purification of prasugrel. Prasugrel can also be purified by converting into acid-addition salt followed by neutralization with a base to produce the substantially pure prasugrel, which can be optionally converted to desired salt. Examples of such acids used for the purification of prasugrel include but are not limited to: inorganic acids such as hydrochloric acid, sulfuric acid, phosphoric acid, hydrobromic acid, or the like; and organic acids such as oxalic acid, maleic acid, fumaric acid, malic acid, tartaric acid, citric acid, benzoic acid, methanesulfonic acid, p-toluenesulfonic acid or the like. Any of the bases listed in step a), can be used for the conversion of acid-addition salt into freebase of prasugrel.

In embodiments of step b), prasugrel obtained in step b) can be purified by dissolving prasugrel in a solvent and adding anti-solvent like water to obtain pure prasugrel. Any of the solvents listed in step-1 can be used for the dissolution of prasugrel. In embodiments, a solution of prasugrel can be combined with any suitable anti-solvent. In a suitable anti-solvent, prasugrel has lower solubility, compared to the solubility of prasugrel in the solvent. An anti-solvent generally will have less polarity than the polarity of the solvent. Examples of anti-solvents include, but are not limited to: saturated or unsaturated, linear or branched, cyclic or acyclic, C₁ to C₁₀ hydrocarbons, such as n-heptane, cyclohexane, methylcyclohexane; water and any mixtures thereof. In specific embodiments prasugrel can be purified by dissolving prasugrel in acetonitrile and adding water as an anti-solvent to the solution. The nature of the solvent, solvent ratios, heating temperatures or heating rates, maintenance time, cooling temperature or cooling rate, and drying conditions also play a significant role in the purity of the prasugrel obtained after the purification. The methods known in the art or the methods described above can be used for the isolation and drying of the prasugrel after purification.

If the prasugrel obtained is an acid-addition salt after purification, then it may be converted to its freebase by neutralization with a base by the procedures known in the art or as described above. In embodiments of step b), prasugrel or salt thereof can be isolated or converted into prasugrel hydrochloride without isolation i.e. in situ.

In embodiments of step c), the prasugrel is converted into prasugrel hydrochloride in the presence of a source of hydrogen chloride in an organic solvent. In embodiments of step c), prasugrel can be dissolved in any suitable inert solvent. Suitable inert solvent can be any solvent which has no adverse effect on the reaction and it can dissolve the starting material to some extent. Examples of such solvents include but are not limited to aliphatic hydrocarbon solvents such as hexane, cyclohexane, heptane, or petroleum ether; aromatic hydrocarbon solvents such as toluene or xylene; halogenated hydrocarbon solvents such as dichloromethane, chloroform, carbon tetrachloride, 1,2-dichloroethane, chlorobenzene or dichlorobenzene; ether solvents such as diethyl ether, diisopropyl ether, tetrahydrofuran, dioxane, or dimethoxyethane; ketone solvents such as acetone, methyl ethyl ketone, methyl isobutyl ketone or diethyl ketone; ester solvents such as ethyl acetate, propyl acetate or butyl acetate; alcohol solvents such as methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, or C₁-C₆alcohols; carboxylic acid solvents such as acetic acid or propionic acid; or nitrile solvents such as acetonitrile or propionitrile; mixture of the said solvents; or the mixture of the said solvents with water. Prasugrel solution can also be obtained from the reaction mass of the previous stage.

In embodiments of step c), solution of prasugrel can be prepared at any suitable temperatures, such as from about 0° C. to about the reflux temperature of the solvent, or about 0° C. to about 80° C. In embodiments of step c), prasugrel solution obtained above can be added by drops in a single lot or in parts to a source of hydrogen chloride. In embodiments of step c), a source of hydrogen chloride can be added or introduced into a prasugrel solution. Alternatively, in embodiments of step c), a source of hydrogen chloride can be added by drops in a single lot or in parts to prasugrel solution.

In embodiments of step c), a source of hydrogen chloride can be hydrochloric acid such as concentrated hydrochloric acid or aqueous diluted hydrochloric acid, hydrogen chloride gas, a solution of hydrogen chloride dissolved in a solvent such as methanol hydrochloride, ethanol hydrochloride, isopropyl alcohol hydrochloride, an acid chloride such as acetyl chloride or quaternary ammonium salts such as ammonium chloride. In embodiments of step c), the concentration of hydrochloric acid dissolved in a solvent can be used from 1% to 40% (w/v). In embodiments of step c), the concentration of hydrochloric acid dissolved in a solvent can be from 1% to 30% (w/v). In embodiments of step c), the concentration of hydrochloric acid dissolved in a solvent can be from 1% to 20% (w/v).

In embodiments of step c), the addition of prasugrel solution or a source of hydrogen chloride to the other can be done at any suitable temperatures, such as from about −10° C. to about 80° C. or from about 0° C. to about 60° C. In embodiments of step c), optionally the seed crystal of prasugrel hydrochloride in any desired polymorphic form can be added before the initiation of the solid formation to the reaction mass prepared above. In embodiments of step c), the reaction mass obtained can be maintained for a time from about 1 hour to about 20 hours, or longer. In embodiments of step c), prasugrel hydrochloride produced in the reaction can be isolated using techniques such as decantation, filtration by gravity or suction, centrifugation, or evaporation of solvent or the like, and optionally washing the resulting solid with a solvent. In one embodiment, the washing is with the solvent used in the above reaction.

In embodiments of step c), the prasugrel hydrochloride obtained may be optionally dried according to the procedures known in the art. In embodiments of step c), prasugrel hydrochloride obtained can be optionally purified by any method known in the art such as recrystallization involving single solvent, mixture of solvents or solvent-anti solvent technique; reprecipitation; slurring in a solvent; or chromatography to improve its chemical purity. Any of the solvents listed in step c), can be used for the purification of prasugrel hydrochloride. The nature of the solvent, solvent ratios, heating temperatures or heating rates, maintenance time, cooling temperature or cooling rate, and drying conditions also play a significant role in the purity of the prasugrel hydrochloride obtained after the purification. In embodiments of step c), prasugrel hydrochloride produced in the reaction can be isolated using techniques such as decantation, filtration by gravity or suction, centrifugation, or evaporation of the solvent or the like, and optionally washing the resulting solid with a solvent. In one embodiment, the washing is with the same solvent used in the purification.

In embodiments of step c), prasugrel hydrochloride that is isolated can be dried at suitable temperatures, such as from about 40° C. to about 100° C. and suitable time from about 1 hour to about 15 hours or longer, using drying equipment known in the art, such as a tray dryer, vacuum oven, air oven, fluidized bed dryer, spin flash dryer, flash dryer, or the like. Drying temperatures and times will be sufficient to achieve desired product purity. In embodiments of step c), isolated prasugrel hydrochloride can be in a crystalline, amorphous, and hydrated or solvated form. In embodiments of step c), isolated prasugrel hydrochloride can be any of the polymorphic forms known in the art such as Form A, Form B1, or Form B2.

In embodiments of step c), isolated prasugrel hydrochloride has a polymorphic form having a PXRD pattern with characteristic peaks located at about 8.1, 13.6, 14.6, 22.1, 25.6 and 26.0±0.2° 2θ (2-theta values). In an embodiment, the processes described in this application produce prasugrel or prasugrel hydrochloride or any prasugrel salts with a content of less than 300 ppm of impurities like diacetone alcohol and mesityl oxide. In an embodiment, the solvents used in the process for the preparation of prasugrel or its pharmaceutically acceptable salts are within the ICH limits in prasugrel hydrochloride.

In an embodiment, it is observed that prasugrel or its pharmaceutically acceptable salts degrades during the storage and leads to impurities such as 5-(2-cyclopropyl-1-(2-fluorophenyl)-2-oxoethyl)-5,6,7,7a-tetrahydrothieno[3,2-c]pyridin-2(4H)-one (OXTP impurity). In another embodiment, it was found that prasugrel or prasugrel hydrochloride when stored in presence of desiccant have a greater stability with respect to impurities such as 5-(2-cyclopropyl-1-(2-fluorophenyl)-2-oxoethyl)-5,6,7,7a-tetrahydrothieno[3,2-c]pyridin-2(4H)-one (OXTP impurity).

The X-ray powder diffraction patterns described herein were generated using a Bruker AXS D8 Advance powder X-ray diffractometer, with a copper K-alpha radiation source. Generally, a diffraction angle (2θ) in powder X-ray diffractometry may have an error in the range of ±0.2°. Therefore, the aforementioned diffraction angle values should be understood as including values in the range of about ±0.2°. Accordingly, the present application includes not only crystals whose peak diffraction angles in powder X-ray diffractometry completely coincide with each other, but also crystals whose peak diffraction angles coincide with each other with an error of about ±0.2°. Therefore, in the present specification, the phrase “having a diffraction peak at a diffraction angle (2θ±0.2°) of 8.1°” means “having a diffraction peak at a diffraction angle (2θ) of 7.9° to 8.3°. Although the intensities of peaks in the x-ray powder diffraction patterns of different batches of a compound may vary slightly, the peaks and the peak locations are characteristic for a specific polymorphic form. Alternatively, the term “about” means within an acceptable standard error of the mean, when considered by one of ordinary skill in the art. The relative intensities of the PXRD peaks can vary depending on the sample preparation technique, crystal size distribution, various filters used, the sample mounting procedure, and the particular instrument employed. Moreover, instrument variation and other factors can affect the 2-theta values. Therefore, the term “substantially” in the context of PXRD is meant to encompass that peak assignments can vary by plus or minus about 0.2.degree. Moreover, new peaks may be observed or existing peaks may disappear, depending on the type of the machine or the settings (for example, whether a Ni filter is used or not.

A process for the preparation of prasugrel hydrochloride by a method of present application can be illustrated as given below in Scheme 1.

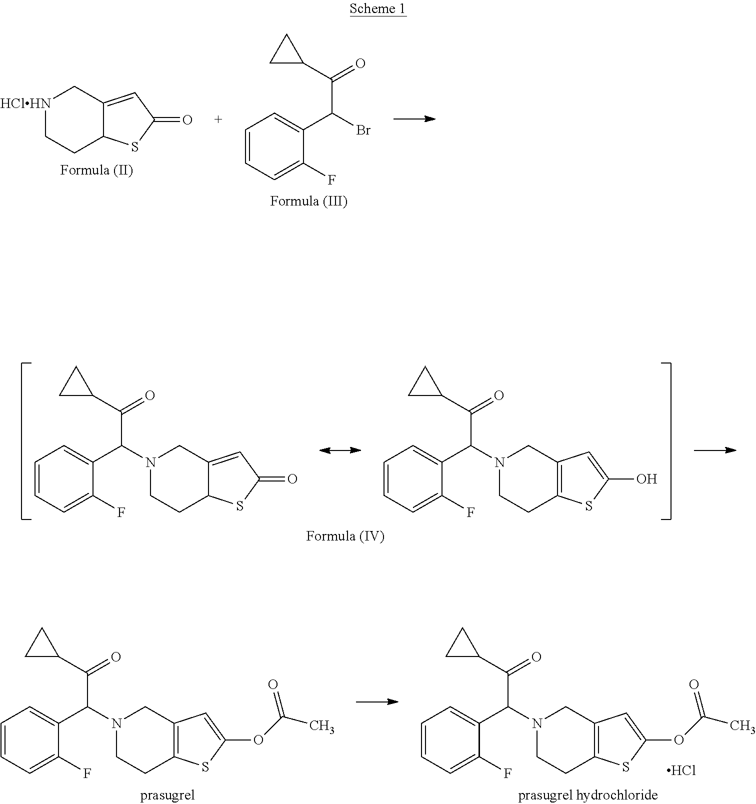

Alternatively, prasugrel hydrochloride can be prepared by a method as given below in Scheme 2.

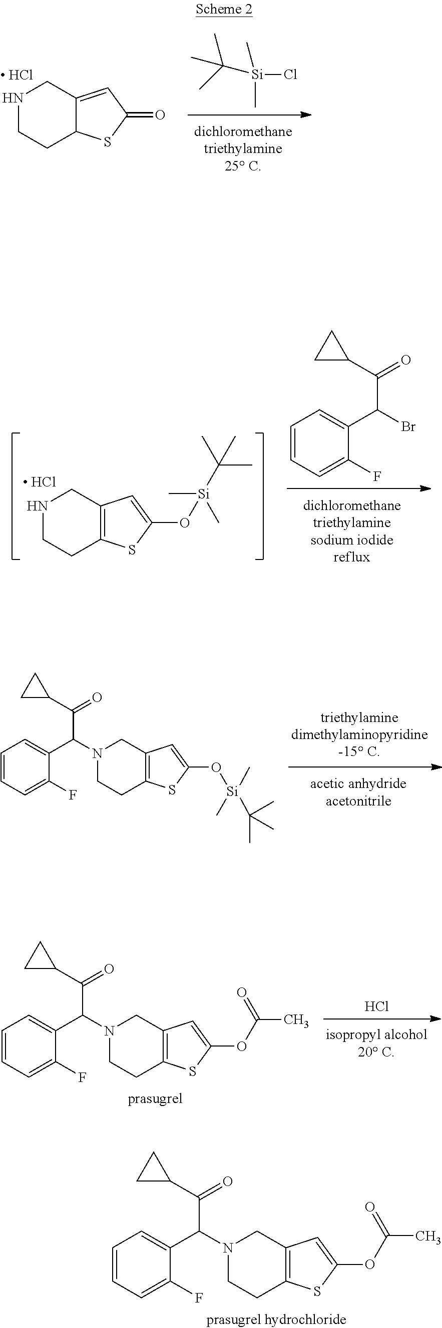

In embodiments, prepared prasugrel or prasugrel hydrochloride can be substantially pure having a chemical purity greater than about 99%, or greater than about 99.5%, or greater than about 99.9%, by weight, as determined using high performance liquid chromatography (HPLC). Prasugrel or prasugrel hydrochloride produced by a method of present application can be chemically pure prasugrel hydrochloride having purity greater than about 99.5% and containing no single impurity in amounts greater than about 0.15%, by HPLC. Prasugrel or prasugrel hydrochloride produced by a method of present application can be chemically pure prasugrel hydrochloride having purity greater than about 99.8% and containing no single impurity in amounts greater than about 0.1%, by HPLC.

Potential impurities possible in prasugrel or prasugrel hydrochloride are the unreacted starting materials and intermediates, described in the present application. In an embodiment, the present method for the preparation of prasugrel hydrochloride from prasugrel produces the impurity i.e., 5-(2-cyclopropyl-1-(2-fluorophenyl)-2-oxoethyl)-5,6,7,7a-tetrahydrothieno[3,2-c]pyridin-2(4H)-one (OXTP impurity) to about a level of less than 0.1%. Possible impurities in prasugrel or its pharmaceutically acceptable salt, in addition to unreacted starting materials or intermediates described in the present application, can have structural formulas as illustrated below.

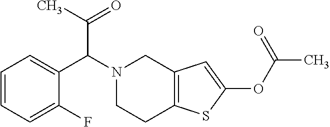 [SMILES: CC(=O)Oc1cc2c(s1)CCN(C(C(C)=O)c1ccccc1F)C2]

5-[(1RS)-2-methyl-1-(2-fluorophenyl)-2-oxoethyl]-4,5,6,7-tetrahydrothieno[3,2-c]pyridin-2-yl acetate (the methyl impurity)

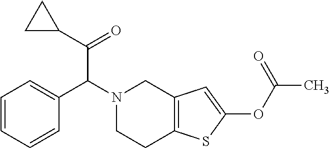 [SMILES: CC(=O)Oc1cc2c(s1)CCN(C(C(=O)C1CC1)c1ccccc1)C2]

5-[(1RS)-2-cyclopropyl-2-oxo-1-phenylethyl]-4,5,6,7-tetrahydrothieno[3,2-c]pyridin-2-yl acetate (the desfluoro impurity)

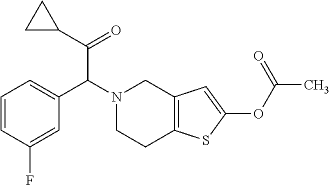 [SMILES: CC(=O)Oc1cc2c(s1)CCN(C(C(=O)C1CC1)c1cccc(F)c1)C2]

5-[(1RS)-2-cyclopropyl-1-(3-fluorophenyl)-2-oxoethyl]-4,5,6,7-tetrahydrothieno[3,2-c]pyridin-2-yl acetate (the 3-fluoro impurity)

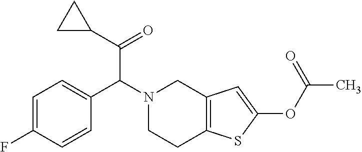 [SMILES: CC(=O)Oc1cc2c(s1)CCN(C(C(=O)C1CC1)c1ccc(F)cc1)C2]

5-[(1RS)-2-cyclopropyl-1-(4-fluorophenyl)-2-oxoethyl]-4,5,6,7-tetrahydrothieno[3,2-c]pyridin-2-yl acetate (the 4-fluoro impurity)

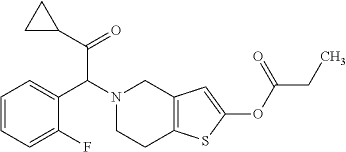 [SMILES: CCC(=O)Oc1cc2c(s1)CCN(C(C(=O)C1CC1)c1ccccc1F)C2]

5-[(1RS)-2-cyclopropyl-1-(2-fluorophenyl)-2-oxoethyl]-4,5,6,7-tetrahydrothieno[3,2-c]pyridin-2-yl propionate (the propionyl impurity)

In another aspect, the application relates to substantially pure prasugrel or its pharmaceutically acceptable salt having less than about 0.1% of the methyl impurity, the propionyl impurity, the desfluoro impurity, the 3-fluoro impurity, the 4-fluoro impurity, or the enantiomer of any impurity. In another aspect, the application provides processes for preparing prasugrel or its pharmaceutically acceptable salt having less than about 0.1% of methyl impurity, propionyl impurity, desfluoro impurity, 3-fluoro impurity, and 4-fluoro impurity or their enantiomers. In another aspect, the application provides prasugrel or its pharmaceutically acceptable salt having less than about 0.1% of 5-[(1RS)-2-cyclopropyl-1-(3-fluorophenyl)-2-oxoethyl]-4,5,6,7-tetrahydrothieno[3,2-c]pyridin-2-yl acetate (3-fluoro impurity) or any of its enantiomer.

In another aspect, the application provides processes for preparing prasugrel or a pharmaceutically acceptable salt having less than about 0.1% of 5-[(1RS)-2-cyclopropyl-1-(3-fluorophenyl)-2-oxoethyl]-4,5,6,7-tetrahydrothieno[3,2-c]pyridin-2-yl acetate (3-fluoro impurity) or any enantiomer of 5-[(1RS)-2-cyclopropyl-1-(3-fluorophenyl)-2-oxoethyl]-4,5,6,7-tetrahydrothieno[3,2-c]pyridin-2-yl acetate comprising:
a) purifying 2-fluoro phenyl acetic acid so the concentration of 3-fluoro phenyl acetic acid is less than about 0.1%; and
b) preparing prasugrel or a pharmaceutically acceptable salt from the 2-fluoro phenyl acetic acid obtained from step a).

In an embodiment, it was found that 3-fluoro impurity may be formed in the prasugrel or its salt when the corresponding impurity (2-bromo-1-cyclopropyl-2-(3-fluorophenyl)ethanone) if present in the compound of formula (III). The removal of 3-fluoro impurity, if present in the prasugrel or its salt may be very difficult as this impurity is having similar solubility. For the removal of 3-fluoro impurity from prasugrel or its salt, it may require successive recrystallization steps or additional purification steps which reduces the yield and enhance the cost as well cycle time. Similarly, if the impurities such as methyl impurity, desfluoro impurity, or 4-fluoro impurity, if present in the prasugrel or its salt, it may require successive recrystallization steps or additional purification steps.

In an embodiment, it was also found that the removal of 2-bromo-1-cyclopropyl-2-(3-fluorophenyl)ethanone from 2-bromo-1-cyclopropyl-2-(2-fluorophenyl)ethanone is very difficult. Similarly, the removal of 2-bromo-1-cyclopropyl-2-phenylethanone and 2-bromo-1-cyclopropyl-2-(4-fluorophenyl)ethanone from 2-bromo-1-cyclopropyl-2-(2-fluorophenyl)ethanone is difficult. Removal of these impurities from 2-bromo-1-cyclopropyl-2-(2-fluorophenyl)ethanone requires additional purification steps.

In an embodiment, it was found that, the source of these impurities is from the starting material i.e., 2-(2-fluorophenyl)acetic acid. If the corresponding impurities i.e., 2-phenyl acetic acid, 2-(4-fluorophenyl)acetic acid and 2-(3-fluorophenyl)acetic acid are controlled at this stage that may lead to the reduced levels of corresponding impurities at 2-bromo-1-cyclopropyl-2-(2-fluorophenyl)ethanone which in turn leads to the reduced levels i.e., less than 0.1% of desfluoro impurity, 3-fluoro impurity, or 4-fluoro impurity at prasugrel or its salt stage.

In an embodiment, it was found that, the 2-(3-fluorophenyl)acetic acid impurity can be controlled to about less than 0.1% in 2-(2-fluorophenyl)acetic acid by purification. The same purification procedure can also controls the other impurities i.e., 2-phenyl acetic acid and 2-(4-fluorophenyl)acetic acid to about a level of less than 0.1% in 2-(2-fluorophenyl)acetic acid by purification. In an embodiment, the application relates to a process for the preparation of substantially pure prasugrel or its pharmaceutically acceptable salt by using the starting material i.e., 2-bromo-1-cyclopropyl-2-(2-fluorophenyl)ethanone having less than 0.1% of 2-bromo-1-cyclopropyl-2-phenylethanone, 2-bromo-1-cyclopropyl-2-(3-fluorophenyl)ethanone and 2-bromo-1-cyclopropyl-2-(4-fluorophenyl)ethanone.

In an embodiment, the application relates to a process for the preparation substantially pure prasugrel or its pharmaceutically acceptable salt by using the starting material i.e., 2-(2-fluorophenyl)acetic acid having less than 0.1% of 2-phenyl acetic acid, 2-(3-fluorophenyl)acetic acid and 2-(4-fluorophenyl)acetic acid. In embodiments of the present application, 2-(2-fluorophenyl)acetic acid may be purified for reducing the impurities 2-phenyl acetic acid, 2-(3-fluorophenyl)acetic acid, or 2-(4-fluorophenyl)acetic acid to about a level of less than about 0.1%.

In embodiments, 2-(2-fluorophenyl)acetic acid may be purified by any method such as recrystallization involving single solvent, mixture of solvents or solvent-anti solvent technique; reprecipitation; slurring in a solvent; or chromatography to improve its chemical purity with different solvents under varying conditions of 2-(2-fluorophenyl)acetic acid-to-solvent ratios, heating temperatures, heating rates, maintenance times, cooling temperatures, cooling rates, and drying conditions or techniques. Any of the solvents listed in step a) of prasugrel, can be used for the purification of 2-(2-fluorophenyl)acetic acid. 2-(2-fluorophenyl)acetic acid can also be purified by converting into its salt followed by neutralization with an acid to produce the free base of 2-(2-fluorophenyl)acetic, which can be optionally converted to desired salt.

Examples of bases used for the salt formation during the purification include but are not limited to: inorganic bases, such as, for example, alkali metal hydrides, such as, for example, lithium hydride, sodium hydride, potassium hydride, or the like; sodamide; n-butyl lithium; lithium diisopropylamide; alkali metal hydroxides, such as, for example, lithium hydroxide, sodium hydroxide, potassium hydroxide, or cesium hydroxide; alkaline metal hydroxides, such as, for example, barium hydroxide, magnesium hydroxide, calcium hydroxide, or the like; alkali metal carbonates, such as, for example, sodium carbonate, potassium carbonate, lithium carbonate, cesium carbonate, or the like; alkaline earth metal carbonates, such as, for example, magnesium carbonate, calcium carbonate, or the like; alkali metal bicarbonates, such as, for example, lithium bicarbonate, sodium bicarbonate, potassium bicarbonate, or the like; and ion exchange resins including resins bound to ions, such as, for example, sodium, potassium, lithium, calcium, magnesium, substituted or unsubstituted ammonium ions, or the like; or organic bases such as methylamine, ethylamine, diethylamine, triethylamine, diisopropylamine, diisopropylethylamine, cyclohexylamine or the like.

The nature of the solvent, solvent ratios, heating temperatures or heating rates, maintenance time, cooling temperature or cooling rate, and drying conditions also play a significant role in the purity of the 2-(2-fluorophenyl)acetic acid or a salt thereof obtained after the purification. The methods known in the art or any of the methods described in the present application, can be used for the isolation and drying of the compound of 2-(2-fluorophenyl)acetic acid or a salt thereof. If the 2-(2-fluorophenyl)acetic acid is a salt after purification, then it may be converted to its freebase by neutralization with acids by the procedures known in the art or by the use of any acids described in the present application.

The 2-(2-fluorophenyl)acetic acid or salt thereof can be substantially pure having a chemical purity greater than about 96%, or greater than about 98%, or greater than about 99%, by weight, as determined using high performance liquid chromatography (HPLC) having less than 0.1% of 2-phenyl acetic acid, 2-(3-fluorophenyl)acetic acid, or 2-(4-fluorophenyl)acetic acid.

The purified 2-(2-fluorophenyl)acetic acid or salt thereof may be converted to 2-bromo-1-cyclopropyl-2-(2-fluorophenyl)ethanone by the methods known in the art or by the methods described herein. The intermediates obtained may be optionally purified, to enhance the chemical purity or to reduce the impurities.

In an embodiment, the substantially pure 2-(2-fluorophenyl)acetic acid may be converted to 2-bromo-1-cyclopropyl-2-(2-fluorophenyl)ethanone through the intermediate 1-cyclopropyl-2-(2-fluorophenyl)ethanone. In an embodiment, the substantially pure 2-(2-fluorophenyl)acetic acid may be converted to 2-bromo-1-cyclopropyl-2-(2-fluorophenyl)ethanone without the isolation of the intermediate 1-cyclopropyl-2-(2-fluorophenyl)ethanone. In an embodiment, intermediate 1-cyclopropyl-2-(2-fluorophenyl)ethanone is prepared by reacting 2-(2-fluorophenyl)acetic acid with ethyl cyclopropanecarboxylate using Grignard reagent in a suitable solvent. The solvents can be used in the reaction but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, or the like.

In another aspect, the application provides a process comprising:
a) purifying 2-fluorophenyl acetic acid so that the concentration of 3-fluoro phenyl acetic acid is less than about 0.1%;
b) reacting the pure 2-fluorophenyl acetic acid obtained in step a), with ethyl cyclopropanecarboxylate to provide 1-cyclopropyl-2-(2-fluorophenyl) ethanone; and
c) brominating, with a suitable brominating agent, the 1-cyclopropyl-2-(2-fluorophenyl)ethanone obtained in step b), to provide 2-bromo-1-cyclopropyl-2-(2-fluorophenyl)ethanone, having less than about 0.1% of 2-bromo-1-cyclopropyl-2-(3-fluorophenyl)ethanone.

The process further comprising converting the 2-bromo-1-cyclopropyl-2-(2-fluorophenyl)ethanone into prasugrel or a pharmaceutically acceptable salt thereof.

In an embodiment the intermediate 1-cyclopropyl-2-(2-fluorophenyl)ethanone is brominated in a suitable solvent to produce 2-bromo-1-cyclopropyl-2-(2-fluorophenyl)ethanone. Any of the solvent used in the above step can be used for bromination. The brominating agent can be used but are not limited to N-bromosuccinimide, bromine, hydrobromic acid, 1,3-dibromo-5,5-dimethylhydantoin (DBDMH), or the like. In an embodiment, 2-bromo-1-cyclopropyl-2-(2-fluorophenyl)ethanone is prepared by reacting 2-(2-fluorophenyl)acetic acid with Ethyl cyclopropanecarboxylate using Grignard reagent to produce intermediate 1-cyclopropyl-2-(2-fluorophenyl)ethanone which is in situ brominated by using a suitable brominating agent. In an embodiment, 2-bromo-1-cyclopropyl-2-(2-fluorophenyl)ethanone is isolated by evaporation of solvent or the like. In an embodiment, any of the intermediates described herein may be purified by the procedures known in the art. In an embodiment, any of the intermediates described herein may be in the state of crystalline, amorphous, hydrate, solvate, or anhydrous form.

In an embodiment, the purified 2-fluoro phenyl acetic acid is used to produce 2-bromo-1-cyclopropyl-2-(2-fluorophenyl)ethanone. The 2-bromo-1-cyclopropyl-2-(2-fluorophenyl)ethanone produced from purified 2-fluoro phenyl acetic acid is used for the preparation of prasugrel or its pharmaceutically acceptable salt. The prasugrel or its pharmaceutically acceptable salt produced by using pure 2-fluoro phenyl acetic acid contains less than 0.1% of the methyl impurity, the propionyl impurity, the desfluoro impurity, the 3-fluoro impurity, the 4-fluoro impurity, or any enantiomer of an impurity.

In another aspect, the application provides a process comprising:
a) preparing a solution of prasugrel in a solvent;
b) adding a solution of hydrogen chloride dissolved in a solvent to the solution of step a);
c) optionally, adding a seed crystal of prasugrel hydrochloride to the reaction mass prepared in step b);
d) isolating the precipitated prasugrel hydrochloride; and
e) optionally, purifying prasugrel hydrochloride to obtain a more purified prasugrel hydrochloride.

In another aspect, the application provides a process comprising:
a) reacting 2-fluoro phenyl acetic acid with an amine to produce an ammonium salt; and
b) converting the ammonium salt obtained in step a), into 2-fluoro phenyl acetic acid that contains less than 0.1% of 2-phenyl acetic acid, 2-(3-fluorophenyl)acetic acid, or 2-(4-fluorophenyl)acetic acid.

Any of the processes described in the present application or any of the methods of known in the art can be used for the preparation of prasugrel or its pharmaceutically acceptable salt starting from purified 2-fluoro phenyl acetic acid. The purified 2-fluoro phenyl acetic acid according to the present application contains less than 0.1% of 2-phenyl acetic acid, 2-(3-fluorophenyl)acetic acid, or 2-(4-fluorophenyl)acetic acid.

In another aspect, the application provides a process comprising:
a) measuring the level of 3-fluoro phenyl acetic acid in one or more batches of 2-fluoro phenyl acetic acid;
b) selecting a batch having less than about 0.1% of 3-fluoro phenyl acetic acid;
c) and synthesizing prasugrel or a salt thereof, having less than 0.1% of 5-[(1RS)-2-cyclopropyl-1-(3-fluorophenyl)-2-oxoethyl]-4,5,6,7-tetrahydrothieno[3,2-c]pyridin-2-yl acetate from the batch of step b).

In another aspect, the application provides a process for preparing prasugrel or a pharmaceutically acceptable salt having less than about 0.1% of the desfluoro impurity, the 3-fluoro impurity, the 4-fluoro impurity, or any enantiomer of an impurity comprising:
a) purifying 2-fluoro phenyl acetic acid so the concentration of 3-fluoro phenyl acetic acid, 4-fluoro phenyl acetic acid, or 2-phenyl acetic acid is less than about 0.1%; and
b) preparing the prasugrel or its pharmaceutically acceptable salt involving the use of 2-fluoro phenyl acetic acid obtained from step a).

Prasugrel hydrochloride is a BCS class 2 drug and particle sizes of prasugrel hydrochloride can have significant effect on dissolution of the product. Prasugrel hydrochloride obtained according to the process of the present application can be milled or micronized by any process known in the art, such as ball milling, jet milling, wet milling etc., to produce a desired particle size distribution. Particle size distributions can be determined using any means, including laser light diffraction equipment sold by Malvern Instruments limited, Malvern, Worcestershire, United Kingdom, Coulter counters, microscopic procedures, etc. The term d(x) means that a particular fraction has particles with a maximum size being the value given; 0.5 represents 50% of the particles and 0.9 represents 90% of the particles.

In another aspect of the application prasugrel obtained according to certain processes of the present application has a particle size distribution wherein: mean particle size is less than about 200 μm or less than about 100 μm; d(0.5) is less than about 200 μm or less than about 25 μm; and d(0.9) is less than about 250 μm or less than about 50 μm. In another aspect of the application prasugrel obtained by the processes herein described, having a mean particle size of about less than about 100 μm. In another aspect of the application prasugrel hydrochloride obtained according to certain processes of the present application has a particle size distribution wherein: mean particle size is less than about 200 μm or less than about 100 μm; d(0.5) is less than about 200 μm or less than about 25 μm; and d(0.9) is less than about 250 μm or less than about 50 μm. In another aspect of the application prasugrel hydrochloride obtained by the processes herein described, having a mean particle size of about 5 μm to about 80 μm, preferably of about 10 μm to about 50 μm.

In another aspect of the application prasugrel hydrochloride obtained by the processes herein described, having a specific surface area of less than 5 m²/g or about 0.5 m²/g to about 5 m²/g as measured by B.E.T. (Brunauer-Emmett-Teller), preferably from about 0.5 m²/g to about 3 m²/g.

In another aspect of the application prasugrel hydrochloride obtained by the processes herein described, having bulk density of about 0.1 to 1.0, preferably from about 0.1 to 0.6 and tapped bulk density 0.1 to 1.0, preferably from about 0.2 to 0.8. In another aspect of the application prasugrel hydrochloride obtained by the processes herein described, having bulk volume of about less than 5 mL/g or from about 0.5 mL/g to about 5 mL/g, preferably from about 1 mL/g to about 4 mL/g and Hausner ratio of about 1 to about 5, preferably from about 1 to about 4.

In another aspect of the application prasugrel hydrochloride obtained by the processes herein described, having compressibility index of about 10% to 100%, preferably from about 20% to about 80%. In another aspect of the application the crystal particles of prasugrel or prasugrel hydrochloride obtained by the processes herein described, can be in needle shape, rod shape, and flake shape or in any other regular shape or mixture of the shapes.

An aspect of the present application provides pharmaceutical compositions containing a therapeutically effective amount of prasugrel hydrochloride together with one or more pharmaceutically acceptable excipients. The pharmaceutical compositions comprising prasugrel hydrochloride of the application together with one or more pharmaceutically acceptable excipients may be formulated as: solid oral dosage forms, such as, but not limited to, powders, granules, pellets, tablets, or capsules; liquid oral dosage forms such as but not limited to syrups, suspensions, dispersions, or emulsions; or injectable preparations such as, but not limited to, solutions, dispersions, or freeze-dried compositions. Formulations may be in the form of immediate release, delayed release, or modified release. Further, immediate release compositions may be conventional, dispersible, chewable, mouth dissolving, or flash melt preparations, and modified release compositions may comprise hydrophilic or hydrophobic, or combinations of hydrophilic and hydrophobic, release rate-controlling substances to form matrix or reservoir systems, or combinations of matrix and reservoir systems. The compositions may be prepared using any one or more of techniques such as direct blending, dry granulation, wet granulation, or extrusion and spheronization. Compositions may be presented as uncoated, film coated, sugar coated, powder coated, enteric coated, or modified release coated.

Pharmaceutically acceptable excipients that are useful in the present application include, but are not limited to, any one or more of: diluents such as starch, pregelatinized starch, lactose, powdered cellulose, microcrystalline cellulose, dicalcium phosphate, tricalcium phosphate, mannitol, sorbitol, sugar, or the like; binders such as acacia, guar gum, tragacanth, gelatin, polyvinylpyrrolidones, hydroxypropyl celluloses, hydroxypropyl methylcelluloses, pregelatinized starches, or the like; disintegrants such as starch, sodium starch glycolate, pregelatinized starches, crospovidones, croscarmellose sodium, colloidal silicon dioxide, or the like; lubricants such as stearic acid, magnesium stearate, zinc stearate, or the like; glidants such as colloidal silicon dioxide or the like; solubility or wetting enhancers such as anionic or cationic or neutral surfactants; complex forming agents such as various grades of cyclodextrins and resins; and release rate controlling agents such as hydroxypropyl celluloses, hydroxymethyl celluloses, hydroxypropyl methylcelluloses, ethylcelluloses, methylcelluloses, various grades of methyl methacrylates, waxes, or the like. Other pharmaceutically acceptable excipients that are useful include, but are not limited to, film-formers, plasticizers, colorants, flavoring agents, sweeteners, viscosity enhancers, preservatives, antioxidants, or the like.

Certain specific aspects and embodiments of the present application will be explained in greater detail with reference to the following examples, which are provided only for purposes of illustration and should not be construed as limiting the scope of the invention in any manner.

### DEFINITIONS

The following definitions are used in connection with the compounds of the present application unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions or the like. The term “reacting” is intended to represent bringing the chemical reactants together under condition such to cause the chemical reaction indicated to take place. The term “prasugrel” is intended to represent the free base of prasugrel. The following abbreviations and acronyms are used herein and have the indicated definitions: HPLC is high-pressure liquid chromatography and RT is retention time. Hyflow is flux-calcined diatomaceous earth treated with sodium carbonate. Hyflo Super Cel® is a registered trademark of the Manville Corp.

An “acetylating agent” is an activated form of acetic acid, which is capable of transferring an acetyl group (CH₃C(O)—) to a substrate. Examples of an “acetylating agent” include, but are not limited to, acetic acid/mineral acid; acetic acid/coupling agent such as DEAD/CAT; acetyl halides such as acetyl fluoride, acetyl chloride, or acetyl bromide; acetic anhydride; mixed anhydrides of acetic acid such as acetic (isobutyl carbonic) anhydride; activated acetic acid an ester solvent like isopropenyl acetate, vinyl acetate, acetic acid N-hydroxysuccinimide ester, or pentafluorophenyl acetate; ketene; or acetyl azide.

An “amide solvent” is an organic solvent containing a carbonyl group (C═O) or phosphorus atom bonded to a nitrogen atom. “Amide solvents” include, but are not limited to, N,N-dimethylformamide (DMF), N,N-dimethylacetamide (DMA), N-methylpyrrolidone (NMP), formamide, acetamide, propanamide, 1,1,3,3-tetramethyl urea, N,N′-dimethylpropylene urea, hexamethyl phosphoramide (HMPA), or hexamethyl phosphorus triamide (HMPT).

“Alcohols” are organic solvents containing a carbon bound to a hydroxyl group. “C₁-C₆Alcohols” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, phenol, glycerol, or the like.

An “aliphatic or aromatic hydrocarbon” is a liquid hydrocarbon, which may be linear, branched, or cyclic and may be saturated, unsaturated, or aromatic. It is capable of dissolving a solute to form a uniformly dispersed solution. Examples of “C₅-C₈ aliphatic or aromatic hydrocarbon solvent”, include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, petroleum ethers, benzene toluene, ethylbenzene, m-xylene, o-xylene, p-xylene, trimethylbenzene, chlorobenzene, fluorobenzene, trifluorotoluene, anisole, or mixtures thereof.

An “brominating agent” is reagent, which is capable of replacing a hydrogen atom on a substrate with a bromine atom. Examples of an “brominating agent” include, but are not limited to, N-bromosuccinimide, bromine, hydrobromic acid, 1,3-dibromo-5,5-dimethylhydantoin (DBDMH), bromine-pyridine complex, N-bromoacetamide; 1-bromo-3-chloro-5,5-dimethylhydantoin, 3-bromo-4,4-dimethyl-2-oxazolidinone, 1-bromo-5,5-dimethylhydantoin, 3-bromo-5,5-dimethylhydantoin, dibromo isocyanuric acid, N-bromoacetamide monohydrate, N-bromocaprolactam, N-bromophthalimide, 3-bromo-1-chloro-5,5-dimethylhydantoin, dibromo isocyanuric acid potassium salt, N-bromo glutarimide, 1,3-dibromo-5-ethyl-5-methylhydantoin, 1-bromo-3,5-dimethyl-5-ethylhydantoin, 1,3-dibromohydantoin, 1,3-dibromo-5-isopropyl-5-methylhydantoin, dibromo cyanoacetamide, or 3-bromo-5-methyl-5-phenyl-imidazolidine-2,4-dione.

An “ester” is an organic solvent containing a carboxyl group —(C═O)—O— bonded to two other carbon atoms. “C₃-C₆ Esters” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, or the like.

An “ether” is an organic solvent containing an oxygen atom —O— bonded to two other carbon atoms. “C₂₋₆Ether solvents” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, or the like.

A “halogenated hydrocarbon” is an organic solvent containing a carbon bound to a halogen. “Halogenated hydrocarbon solvent” include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

A “ketone” is an organic solvent containing a carbonyl group —(C═O)— bonded to two other carbon atoms. “C₃₋₆Ketones” include, but are not limited to, acetone, ethyl methyl ketone, diethyl ketone, methyl isobutyl ketone, or the like.

A “nitrile” is an organic solvent containing a cyano —(C≡N) bonded to another carbon atom. “C₂₋₆ Nitrile solvents” include, but are not limited to, acetonitrile, propionitrile, butanenitrile, or the like.

An “organic base” is an organic compound, which acts as a base. Examples of such bases include, but are not limited to, triethylamine, diisopropylamine, Hunig's base, DABCO, triethanolamine, tributylamine, pyridine, lutidine, 4-dimethylamino pyridine (DMAP), N-methylpyrrolidine, diethanolamine, 4-methylmorpholine, dimethylethanolamine, tetramethylguanidine, morpholine, imidazole, 2-methylimidazole, 4-methylimidazole, tetramethylammonium hydroxide, tetraethylammonium hydroxide, N-methyl-1,5,9-triazabicyclo[4.4.0]decene, 1,8-diazabicyclo[5.4.0]undec-7-ene, dicyclohexylamine, and picoline.

Representative “pharmaceutically acceptable acids” include, but are not limited to, those capable of making water-soluble and water-insoluble salts, such as the acetate, benzenesulfonate, benzoate, bicarbonate, bisulfate, bitartrate, borate, butyrate, camsylate (camphorsulfonate), carbonate, citrate, clavulariate, dihydrochloride, diphosphate, edisylate (camphorsulfonate), esylate (ethanesulfonate), fumarate, gluceptate (glucoheptonate), gluconate, glucuronate, glutamate, hexafluorophosphate, hexylresorcinate, hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, malate, maleate, mandelate, mesylate, mucate, napsylate, nitrate, oleate, oxalate, palmitate, pamoate (4,4′-methylenebis-3-hydroxy-2-naphthoate, or embonate), pantothenate, phosphate, picrate, propionate, p-toluenesulfonate, salicylate, stearate, subacetate, succinate, sulfate, sulfosaliculate, suramate, tannate, tartrate, and valerate salts.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present application.

EXAMPLES

### Example 1

### Purification of 2-(2-fluorophenyl)acetic acid

2-(2-Fluorophenyl) acetic acid (5 g) (containing 0.16% 2-(3-fluorophenyl)acetic acid impurity) and isopropyl alcohol (20 mL) are charged into a round bottom flask at 26° C. The ammonia gas is passed into the reaction mass to adjust the pH in the range of 8 to 9. The reaction mass is cooled to 4° C. and stirred at 4° C. to 5° C. for 2 hours. The solid is collected by filtration, washed with chilled isopropyl alcohol (10 mL), and then dried under vacuum at 58° C. for about 2 hours. Yield: 4.9 g. HPLC Purity: 99.93%, 2-(3-fluorophenyl)acetic acid as ammonium salt impurity: 0.07%. The solid material (4.5 g) and water (15 mL) are charged into a round bottom flask at 26° C. Concentrated hydrochloric acid (4 mL) is added to reaction mass at 26° C. and stirred at 26-27° C. for 1 hour. The solid is collected by filtration, washed with water (5 mL), and then dried under vacuum at 35° C. for 2 hours. Yield: 3.7 g, HPLC Purity: 99.97%, 2-(3-fluorophenyl)acetic acid impurity: 0.03%.

### Example 2

### Preparation of 2-bromo-1-cyclopropyl-2-(2-fluorophenyl)ethanone

Tetrahydrofuran (90 mL), magnesium turnings (4.4 g), and iodine are charged into a round bottom flask under nitrogen atmosphere at 28° C. A solution of 2-bromopropane (22.07 g) in tetrahydrofuran (60 mL) is added to the reaction mass at 20° C. in 30 minutes and stirred at the same temperature for 1 hour. The solution of 2-(2-fluoro phenyl)acetic acid (11 g) [having less than 0.06% of 2-(3-fluorophenyl)acetic acid impurity] in tetrahydrofuran (20 mL) is added to the reaction mass at 20° C. in 30 minutes. The reaction mass is heated to 65° C. and stirred at the same temperature for 3 hours. The reaction mass is cooled to 5° C. Ethyl cyclopropanecarboxylate (7.8 g) is added to the reaction mass in 10 minutes at 5° C. The reaction mass is heated to 70° C. and stirred at the same temperature for 3 hours. The reaction mass is cooled to 20° C. Water (10 mL) and 2N hydrochloric acid (90 mL) are added to the reaction mass at 20° C. and stirred for 20 minutes. The organic and aqueous layers are separated. The aqueous layer is extracted with dichloromethane twice (10 mL×2). The combined organic layer is washed with saturated solution of sodium bicarbonate. The organic layer is dried over sodium sulfate. The solvent is evaporated under vacuum at 40° C. to get residue (6.8 g). Chloroform (130 ml) is added to the residue at 28° C. N-bromo succinamide (7.8 g), azobisisobutyronitrile (0.39 g) and p-toluenesulfonic acid (0.19 g) are added to the reaction mass at 28° C. The reaction mass is heated to 65° C. and stirred at the same temperature for 4 hours. The reaction mass is cooled to 5° C. and stirred at the same temperature for 1 hour. The reaction mass is filtered at 5° C. to remove unwanted residue if any. The filtrate is washed with aqueous solution of sodium bisulfate (50 mL). The organic and aqueous layers are separated. The aqueous layer is extracted with chloroform (10 mL). The organic layers are combined and evaporated at 65° C. to get residue. Yield: 18.2 g; 2-bromo-1-cyclopropyl-2-(3-fluorophenyl)ethanone impurity: 0.051% by HPLC.

### Example 3

### Preparation of 5-(2-cyclopropyl-1-(2-fluorophenyl)-2-oxoethyl)-4,5,6,7-tetrahydrothieno[3,2-c]pyridin-2-yl acetate

2-Bromo-1-cyclopropyl-2-(2-fluorophenyl)ethanone (7.5 g) (obtained from example 2 and acetonitrile (25 mL) are charged into a round bottom flask at 26° C. Sodium carbonate (6.06 g) is added to the reaction mass at 26° C. 5,6,7,7a-tetrahydrothieno[3,2-c]pyridin-2(4H)-one (5.0 g) is added to the reaction mass at 26° C. The reaction mass is stirred for 28 hours at 26° C. The reaction mass is filtered and washed with acetonitrile (10 mL). The filtrate is charged into a round bottom flask at 27° C. and cooled to 4° C. N-methylmorpholine (5.25 g) and 4-dimethylaminopyridine (0.0349 g) are added to the filtrate at 4° C. Acetic anhydride (3.981 g) is added by drops to the reaction mass at 4° C. The reaction mass is stirred for 3 hours at 4° C. Water (35 ml) is added by drops to the reaction mass at 4° C. in 15 minutes. The reaction mass is stirred at 4° C. for 1 hour 30 minutes. The solid is collected by filtration and washed with chilled acetonitrile and water mixture (5+5 ml). The wet solid material and acetonitrile (25 mL) are charged into a round bottom flask at 28° C. and heated to 48° C. for 10 minutes to obtain clear solution. The solution is cooled to 5° C. and stirred at the same temperature for 1 hour 30 minutes. The solid is collected by filtration, washed with chilled acetonitrile (5 mL), and then dried under vacuum at 70° C. for 5 hours. Yield: 3.96 g; HPLC Purity: 99.57%; 3-fluoro impurity: 0.027%.

### Example 4

### Preparation of Prasugrel Hydrochloride

Prasugrel (3 g) (obtained from example 3 and acetone (24 mL) are charged into a round bottom flask at 28° C. and stirred until a clear solution is obtained. Activated carbon (0.06 g) is added to the reaction mass at 30° C. and stirred for 30 minutes at the same temperature. The reaction mass is filtered under vacuum through Hyflow and washed with acetone (6 mL). The filtrate is charged into a round bottom flask at 28° C. and heated to 48° C. for 30 minutes. An isopropyl alcohol solution of hydrochloric acid (2.9 mL, 10%) is added by drops to the reaction mass at 48° C. over 10 minutes. The reaction mass is seeded with prasugrel hydrochloride (0.006 g) at 48° C. and stirred at the same temperature for 1 hour 30 minutes. The reaction mass is stirred for 1 hour 30 minutes at 30° C. The solid is collected by filtration, washed with acetone (3 mL), and then dried under vacuum at 65° C. for 7 hours. Yield: 2.6 g; HPLC Purity: 99.87%; 3-fluoro impurity: 0.037%; any other impurity: less than 0.1%.

### Example 5

### Preparation of 5-(2-cyclopropyl-1-(2-fluorophenyl)-2-oxoethyl)-4,5,6,7-tetrahydrothieno[3,2-c]pyridin-2-yl acetate starting from 2-bromo-1-cyclopropyl-2-(2-fluorophenyl)ethanone having 0.16% of 2-bromo-1-cyclopropyl-2-(3-fluorophenyl)ethanone

2-Bromo-1-cyclopropyl-2-(2-fluorophenyl)ethanone (150.9 g) and acetonitrile (500 mL) are charged into a round bottom flask at 27° C. Sodium carbonate (121.66 g) is added to the reaction mass at 27° C. 5,6,7,7a-tetrahydro-thieno[3,2-c]pyridin-2(4H)-one (100 g) is added to the reaction mass in three equal lots at interval of 20 minutes between addition of each lot at 27° C. The reaction mass is stirred for 12 hours at 27° C. The reaction mass is filtered and washed with acetonitrile (200 mL). The filtrate is charged into a round bottom flask at 27° C. and cooled to 3° C. N-Methylmorpholine (105.5 g) and 4-dimethylaminopyridine (0.69 g) are added to the filtrate at 3° C. Acetic anhydride (79.89 g) is added to the reaction mass at 3° C. over 15 minutes. The reaction mass is stirred for 5 hours at 3° C. Water (700 ml) is added dropwise to the reaction mass at 3° C. in 1 hour 30 minutes. The reaction mass is stirred at 3° C. for 1 hour 30 minutes. The solid is collected by filtration and washed with a chilled acetonitrile and water mixture (100+100 ml). The wet solid material and acetonitrile (500 mL) are charged into a round bottom flask at 26° C. and heated to 47° C. for 30 minutes to obtain clear solution. The solution is cooled to 1° C. and stirred at the same temperature for 2 hours. The solid is collected by filtration, washed with chilled acetonitrile (100 mL), and then dried under vacuum at 60° C. for 5 hours. Yield: 101.8 g; HPLC Purity: 99.68%; [3-fluoro impurity: 0.16% (before purification). 3-fluoro impurity: 0.13% (after purification)].

### Example 6

### Purification of Prasugrel

Prasugrel (33.5 g) and acetonitrile (125 mL) are charged into a round bottom flask at 30° C. The reaction mass is heated to 50° C. to get clear solution. The solution is cooled to 1° C. Water (125 mL) is added to the solution in 30 minutes at 1° C. The reaction mass is maintained for 1 hour 30 minutes at 2° C. The solid is obtained by filtration and washed with mixture of acetonitrile and water (25+25 mL). The material is dried under vacuum at 60° C. for 7 hours. Yield: 28.5 g. HPLC purity: 99.88%, 3-fluoro impurity: 0.03%

### Example 7

### Preparation of 2-(2-((tert-butyldimethylsilyl)oxy)-6,7-dihydrothieno[3,2-c]pyridin-5(4H)-yl)-1-cyclopropyl-2-(2-fluorophenyl)ethanone

Dichloromethane (75 mL) and 5,6,7,7a-tetrahydrothieno[3,2-c]pyridin-2(4H)-one hydrochloride (25 g) and t-butyl dimethyl silyl chloride (21.6 g) are charged into a round bottom flask at 28° C. Triethylamine (14.5 g) is then added dropwise to the reaction mass at 28° C., and the obtained reaction mass is stirred for 1 hour 45 minutes at 30° C. 2-Bromo-1-cyclopropyl-2-(2-fluorophenyl)ethanone (40.93 g), sodium iodide (0.42 g), triethylamine (26.4 g) and dichloromethane (25 mL) are added to the reaction mass at 29° C. The reaction mass is heated at reflux for 4 hours at 44° C. Phosphate buffer solution (75 mL) is then added to the reaction mass at 25° and the reaction mass is stirred at the same temperature for 25 minutes. The organic and aqueous layers are separated. The aqueous layer is extracted with dichloromethane twice (50 mL×2). The solvent is evaporated under vacuum at 40° C. to get residue. Acetonitrile (100 ml) is added to residue at 42° C. and reduce the temperature to 30° C. Water (50 mL) is then added to reaction mass at 30° C. The solution is then cooled to 0° C. and maintained for 1 hour at 0° C. The solid is collected by filtration, washed with chilled acetonitrile and water mixture (25+25 ml), and then dried under vacuum at 55° C. Yield: 40.8 g; HPLC Purity: 97.4%.

### Example 8

### Preparation of 5-(2-cyclopropyl-1-(2-fluorophenyl)-2-oxoethyl)-4,5,6,7-tetrahydrothieno[3,2-c]pyridin-2-yl acetate

Acetonitrile (625 mL) and 2-(2-((tert-butyldimethylsilyl)oxy)-6,7-dihydrothieno[3,2-c]pyridin-5(4H)-yl)-1-cyclopropyl-2-(2-fluorophenyl)ethanone (100 g) are charged into a round bottom flask at 25° C. Triethylamine (37.63 g) and 4-dimethylaminopyridine (0.28 g) are added to the reaction mass at 15° C. The reaction mass is cooled to −4° C. The solution of acetic anhydride (29.9 g) in acetonitrile (37 mL) is added dropwise to the reaction mass at −4° C. in 1 hour 15 minutes. The reaction mass is maintained for 1 hour at −3° C. Water (368 mL) is added to the reaction mass at −5° C. in 45 minutes. The reaction mass is maintained for 45 minutes at −5° C. The solid is collected by filtration, washed with chilled acetonitrile and water mixture (100+100 ml), and then dried under vacuum at 62° C. Yield: 67.7 g; HPLC Purity: 99.63%.

### Example 9

### Conversion of Prasugrel Hydrochloride to Prasugrel

Prasugrel hydrochloride (150 g), acetonitrile (600 mL) and water (600 mL) are charged into a round bottom flask at 28° C. The reaction mass is stirred to obtain a clear solution at 28° C. The solution is cooled to 0 to 5° C. The pH of the solution is adjusted to 8.2 with aqueous sodium bicarbonate (5% w/v) at 0 to 5° C. The reaction mass is stirred for 2 hours at 0 to 5° C. The solid is collected by filtration, washed with acetonitrile and water (150mL+150 mL), and then dried under vacuum at 60° C. for 5 hours. Yield: 132.5 g

### Example 10

### Preparation of Prasugrel Hydrochloride

Prasugrel (100 g) and acetone (800 mL) are charged into a round bottom flask at 25° C. The reaction mass is heated to 35° C., stirred to obtain a clear solution. Basic carbon (0.2 g) is added to the reaction mass at 36° C. and stirred for 30 minutes at the same temperature. The reaction mass is filtered under vacuum through Hyflow and washed with acetone (200 mL). The filtrate is charged into a round bottom flask at 27° C. and heated to 48° C. for 30 minutes. An isopropyl alcohol solution of hydrochloric acid (99.7 mL, 9.8%) is added to the reaction mass at 48° C. in 10 minutes. The reaction mass is seeded with prasugrel hydrochloride (0.2 g) at 48° C. and stirred at the same temperature for 2 hours. The reaction mass is stirred at for 1 hour at 44° C. The solid is collected by filtration at 44° C., washed with acetone (100 mL), and then dried under vacuum at 60° C. for 4 hours. Yield: 99.3 g; HPLC Purity: 99.71%.

### Example 11

### Preparation of Prasugrel Hydrochloride

Prasugrel (10 g) and acetone (100 mL) are charged into a round bottom flask at 28° C. The reaction mass is heated to 42° C., stirred to obtain a clear solution. An isopropyl alcohol solution of hydrochloric acid (5 mL, 10%) is added by drops to the reaction mass at 42° C. over 20 minutes. The reaction mass is seeded with prasugrel hydrochloride (0.02 g) at 42° C. and stirred at the same temperature for 1 hour. An isopropyl alcohol solution of hydrochloric acid (5 mL, 10%) is further added by drop to the reaction mass at 45° C. over 20 minutes. The reaction mass is stirred for 2 hours at 45° C. The solid is collected by filtration at 45° C., washed with acetone (10 mL), and then dried under vacuum at 75° C. for 4 hours. Yield: 9.8 g; HPLC Purity: 99.68%

### Example 12

Preparation of Prasugrel Hydrochloride

Prasugrel (5 g) and methyl ethyl ketone (40 mL) are charged into a round bottom flask at 25° C. and stirred until a clear solution is obtained. Concentrated hydrochloric acid (1.4 mL) is then by drops added to the reaction mass in 15 minutes at 25° C. The reaction mass is stirred for 1 hour 45 minutes at 25° C. The solid is collected by filtration, washed with methyl ethyl ketone (15 mL), and then dried under vacuum at 51° C. Yield: 4.3 g; HPLC Purity: 99.08%.

### Example 13

Preparation of Prasugrel Hydrochloride

Prasugrel (5 g) and methanol (25 mL) are charged into a round bottom flask at 24° C. Methanolic hydrochloric acid (4 mL) (12%) is added by drops to the reaction mass in 15 minutes at 25° C. The reaction mass is stirred for 1 hour 15 minutes at 25° C. Methanol is evaporated completely under vacuum at 50° C. Acetone (50 mL) is then added to the reaction mass at 35° C. The reaction mass is stirred for 1 hour 15 minutes at 25° C. The solid is collected by filtration, washed with acetone (10 mL), and then dried under vacuum at 53° C. Yield: 4.2 g; HPLC Purity: 98.85%.

### Example 14

### Preparation of Prasugrel Hydrochloride

Prasugrel (5 g) and ethyl acetate (50 mL) are charged into a round bottom flask at 30° C. and stirred until a clear solution is obtained. The reaction mass is cooled to 25° C. An ethyl acetate solution of hydrochloric acid (5.5 mL, 9%) is added by drops to the reaction mass in 10 minutes at 25° C. The reaction mass is stirred for 1 hour 30 minutes at 25° C. The solid is collected by filtration, washed with ethyl acetate (10 mL), and then dried under vacuum. Yield: 3.9 g; HPLC Purity: 98.17%.

### Example 15

### Preparation of Prasugrel Hydrochloride Form A from Amorphous Prasugrel Hydrochloride

Prasugrel hydrochloride amorphous (11 g) and acetone (100 mL) are charged into a round bottom flask at 25° C. The reaction mass is stirred at 25° C. to obtain a clear solution. The resulting solution is stirred for 1 hour 30 minutes at 25° C. The prasugrel hydrochloride Form A is collected by filtration at 25° C., washed with acetone (20 mL), and then dried under vacuum at 65° C. for 3 hours 30 minutes. Yield: 8.5 g.

### Example 16

### Preparation of Prasugrel Hydrochloride Form C from Amorphous Prasugrel Hydrochloride

Prasugrel hydrochloride amorphous (11 g) and isopropyl alcohol (100 mL) are charged into a round bottom flask at 25° C. The reaction mass is stirred at 25° C. to obtain a clear solution. The resulting solution is stirred for 1 hour 45 minutes at 25° C. The prasugrel hydrochloride Form C is collected by filtration at 25° C., washed with isopropyl alcohol (20 mL), and then dried under vacuum at 65° C. for 3 hours 30 minutes. Yield: 8.6 g

### Example 17

### Preparation of Amorphous Prasugrel Hydrochloride

Prasugrel (30 g) and dichloromethane (300 mL) are charged into a round bottom flask at 25° C. The reaction mass is stirred to obtain a clear solution. An isopropyl alcohol solution of hydrochloric acid (30 mL, 9.8%) is added by drops to the reaction mass in 15 minutes at 25° C. The reaction mass is stirred for 30 minutes at 25° C. The solvent is evaporated from the reaction mass under vacuum at 40° C. to result solid material which is further dried under vacuum for 3 hours 30 minutes at 40° C. to obtain amorphous prasugrel hydrochloride. Yield: 34.5 g.

Throughout this application, various publications are referenced. The disclosures of these publications in their entireties are hereby incorporated by reference into this application in order to more fully describe the state of the art as known to those skilled therein as of the date of the application described and claimed herein.

While particular embodiments of the present application have been illustrated and described, it would be obvious to those skilled in the art that various other changes and modifications can be made without departing from the spirit and scope of the application. It is therefore intended to cover in the appended claims all such changes and modifications that are within the scope of this application.

1-14. (canceled)

15. A process comprising:
a) reacting 5,6,7,7a-tetrahydro-thieno[3,2-c]pyridin-2(4H)-one or a salt thereof with 2-bromo-1-cyclopropyl-2-(2-fluorophenyl)ethanone in the presence of an inorganic base and in a solvent to produce 5-(2-cyclopropyl-1-(2-fluorophenyl)-2-oxoethyl)-5,6,7,7a-tetrahydrothieno[3,2-c]pyridin-2(4H)-one;
b) acetylating in situ the obtained 5-(2-cyclopropyl-1-(2-fluorophenyl)-2-oxoethyl)-5,6,7,7a-tetrahydrothieno[3,2-c]pyridin-2(4H)-one in step a), into prasugrel by treating with an acetylating agent in the presence of an organic base; and
c) optionally converting the prasugrel of step b), into prasugrel hydrochloride in the presence of a source of hydrogen chloride in an organic solvent.

16. The process of claim 15, wherein the solvent of step a) is an ether solvent, a ketone solvent, an ester solvent, an alcohol solvent, a nitrile solvent, an amide solvent, a sulfoxide solvent, water, or mixtures thereof.

17. The process of claim 15, wherein step b) is carried out without isolating the 5-(2-cyclopropyl-1-(2-fluorophenyl)-2-oxoethyl)-5,6,7,7a-tetrahydrothieno[3,2- c]pyridin-2(4H)-one obtained in step a).

18. The process of claim 15, wherein the acetylating agent of step b) is acetic anhydride.

19. Prasugrel or a pharmaceutically acceptable salt thereof, prepared according to claim 15, containing less than 0.1% 5-[(1RS)-2-cyclopropyl-1-(3-fluorophenyl)-2-oxoethyl]-4,5,6,7-tetrahydrothieno[3,2-c]pyridin-2-yl acetate.

20. Prasugrel or a pharmaceutically acceptable salt thereof, prepared according to claim 15, having less than about 0.1% of the methyl impurity, the propionyl impurity, the desfluoro impurity, the 3-fluoro impurity, the 4-fluoro impurity, or the enantiomer of any impurity.

21. A pharmaceutical composition comprising a pharmaceutically acceptable excipient and the prasugrel hydrochloride of claim 15.

22. A process for preparing prasugrel or a pharmaceutically acceptable salt thereof, having less than about 0.1% of 5-[(1RS)-2-cyclopropyl-1-(3-fluorophenyl)-2-oxoethyl]-4,5,6,7-tetrahydrothieno[3,2-c]pyridin-2-yl acetate or any enantiomer of 5-[(1RS)-2-cyclopropyl-1-(3-fluorophenyl)-2-oxoethyl]-4,5,6,7-tetrahydrothieno[3,2-c]pyridin-2-yl acetate comprising:
a) purifying 2-fluoro phenyl acetic acid so the concentration of 3-fluoro phenyl acetic acid is less than about 0.1%; and
b) preparing the prasugrel or a pharmaceutically acceptable salt from the 2-fluoro phenyl acetic acid obtained from step a).

23. Prasugrel or a pharmaceutically acceptable salt thereof, prepared according to claim 22, containing less than 0.1% 5-[(1RS)-2-cyclopropyl-1-(3-fluorophenyl)-2-oxoethyl]-4,5,6,7-tetrahydrothieno[3,2-c]pyridin-2-yl acetate.

24. Prasugrel or a pharmaceutically acceptable salt thereof, prepared according to claim 22, having less than about 0.1% of the methyl impurity, the propionyl impurity, the desfluoro impurity, the 3-fluoro impurity, the 4-fluoro impurity, or the enantiomer of any impurity.

25. A pharmaceutical composition comprising a pharmaceutically acceptable excipient and the prasugrel hydrochloride of claim 22.

26. A process for preparing prasugrel or a pharmaceutically acceptable salt thereof, having less than about 0.1% of the desfluoro impurity, the 3-fluoro impurity, the 4-fluoro impurity, or any enantiomer of an impurity comprising:
a) purifying 2-fluoro phenyl acetic acid so the concentration of 3-fluoro phenyl acetic acid, 4-fluoro phenyl acetic acid, 2-phenyl acetic acid is less than about 0.1%; and
b) preparing the prasugrel or its pharmaceutically acceptable salt involving the use of 2-fluoro phenyl acetic acid obtained from step a).

27. Prasugrel or a pharmaceutically acceptable salt thereof, prepared according to claim 26, containing less than 0.1% 5-[(1RS)-2-cyclopropyl-1-(3-fluorophenyl)-2-oxoethyl]-4,5,6,7-tetrahydrothieno[3,2-c]pyridin-2-yl acetate.

28. Prasugrel or a pharmaceutically acceptable salt thereof, prepared according to claim 26, having less than about 0.1% of the methyl impurity, the propionyl impurity, the desfluoro impurity, the 3-fluoro impurity, the 4-fluoro impurity, or the enantiomer of any impurity.

29. A pharmaceutical composition comprising a pharmaceutically acceptable excipient and the prasugrel hydrochloride of claim 26.

30. A process comprising:
a) preparing a solution of prasugrel in a solvent;
b) adding a solution of hydrogen chloride dissolved in a solvent to the solution of step a);
c) optionally, adding a seed crystal of prasugrel hydrochloride to the reaction mass prepared in step b);
d) isolating the precipitated prasugrel hydrochloride; and
e) optionally, purifying prasugrel hydrochloride to obtain a more purified prasugrel hydrochloride.

31. Prasugrel or a pharmaceutically acceptable salt thereof, prepared according to claim 30, containing less than 0.1% 5-[(1RS)-2-cyclopropyl-1-(3-fluorophenyl)-2-oxoethyl]-4,5,6,7-tetrahydrothieno[3,2-c]pyridin-2-yl acetate.

32. Prasugrel or a pharmaceutically acceptable salt thereof, prepared according to claim 30, having less than about 0.1% of the methyl impurity, the propionyl impurity, the desfluoro impurity, the 3-fluoro impurity, the 4-fluoro impurity, or the enantiomer of any impurity.

33. A pharmaceutical composition comprising a pharmaceutically acceptable excipient and the prasugrel hydrochloride of claim 30.

34. A process comprising:
a) measuring the level of 3-fluoro phenyl acetic acid in one or more batches of 2-fluoro phenyl acetic acid;
b) selecting a batch having less than about 0.1% of 3-fluoro phenyl acetic acid; and
c) synthesizing prasugrel or a salt thereof, having less than 0.1% of 5-[(1RS)-2-cyclopropyl-1-(3-fluorophenyl)-2-oxoethyl]-4,5,6,7-tetrahydrothieno[3,2-c]pyridin-2-yl acetate from the batch of step b).

35. Prasugrel or a pharmaceutically acceptable salt thereof, prepared according to claim 34, containing less than 0.1% 5-[(1RS)-2-cyclopropyl-1-(3-fluorophenyl)-2-oxoethyl]-4,5,6,7-tetrahydrothieno[3,2-c]pyridin-2-yl acetate.

36. Prasugrel or a pharmaceutically acceptable salt thereof, prepared according to claim 34, having less than about 0.1% of the methyl impurity, the propionyl impurity, the desfluoro impurity, the 3-fluoro impurity, the 4-fluoro impurity, or the enantiomer of any impurity.

37. A pharmaceutical composition comprising a pharmaceutically acceptable excipient and the prasugrel hydrochloride of a claim 34.

38. A process comprising:
a) purifying 2-fluorophenyl acetic acid so that the concentration of 3-fluoro phenyl acetic acid is less than about 0.1%;
b) reacting the pure 2-fluorophenyl acetic acid obtained in step a), with ethyl cyclopropanecarboxylate to provide 1-cyclopropyl-2-(2-fluorophenyl)ethanone; and
c) brominating, with a suitable brominating agent, the 1-cyclopropyl-2-(2-fluorophenyl)ethanone obtained in step b), to provide 2-bromo-1-cyclopropyl-2-(2-fluorophenyl)ethanone, having less than about 0.1% of 2-bromo-1-cyclopropyl-2-(3-fluorophenyl)ethanone.

39. The process of claim 38, further comprising converting the 2-bromo-1-cyclopropyl-2-(2-fluorophenyl)ethanone into prasugrel or a pharmaceutically acceptable salt thereof.

40. A pharmaceutical composition comprising a pharmaceutically acceptable excipient and the prasugrel of claim 39.

41. A process comprising:
a) reacting 2-fluoro phenyl acetic acid with an amine to produce an ammonium salt; and
b) converting the ammonium salt obtained in step a), into 2-fluoro phenyl acetic acid that contains less than 0.1% of 2-phenyl acetic acid, 2-(3-fluorophenyl)acetic acid, or 2-(4-fluorophenyl)acetic acid.
